(19) United States
(12) Patent Application Publication
(10) Pub. No.: US 2025/0362284 A1
(43) Pub. Date: Nov. 27, 2025

(54) METHOD OF TESTING BLOOD

(71) Applicant: National Central University, Taoyuan City (TW)

(72) Inventors: Kun-Yu Lai, Taoyuan City (TW); Fan-Ching Chien, Taoyuan City (TW)

(21) Appl. No.: 18/793,976

(22) Filed: Aug. 5, 2024

(30) Foreign Application Priority Data
May 21, 2024 (TW) 113118815

(51) Int. Cl. G01N 33/49; G01N 1/28; G01N 21/65

(52) U.S. Cl. CPC G01N 33/491; G01N 1/2813; G01N 21/65

(57) ABSTRACT

The present invention provides a blood testing method. Provide a testing substrate. Dispose a blood sample on the testing substrate. The blood sample comprises a plurality of target substances. The testing substrate separates the blood sample to a first part and a second part. Use a spectrometer to detect the Raman spectrum of the first part or the second part on the testing substrate for measuring the properties of the plurality of target substances and generating target spectrum. Then a control unit compares the targe spectrum and the preset spectrum to generate the testing result. The testing substrate comprises a substrate, a quantum well structure, a first-type doped semiconductor layer, a testing surface, and a plurality of nanometer metal particles. The plurality of nanometer metal particles are located in the testing surface.

### FIELD OF THE INVENTION

The present invention relates to a method of testing blood, in particular, a method of separating blood using a testing substrate, amplifying the Raman spectrum of a target substance in the blood, and testing the blood using a spectrometer.

### BACKGROUND OF THE INVENTION

According to the World Health Organization, lung cancer is the primary cause of cancer death worldwide. Although the standardized mortality rate of lung cancer in Taiwan dropped from 26 per 100,000 people in 2011 to 21.8 per 100,000 people in 2022, a decrease of 16.2%, it still ranks first among the causes of cancer death in Taiwan. A total of 10,053 people died of lung cancer in 2022, accounting for one fifth (19.4%) of all cancer deaths.

Lung cancer cases have high mortality and low survival rates, which are mainly related to the stage of diagnosis. In 2021, 5.4% of diagnosed lung cancer cases were diagnosed as stage 0, 29.9% as stage 1, 3.4% as stage 2, 11.1% as stage 3, and 50.2% as stage 4. Currently, the 5-year survival rate for stage 1 lung cancer is 90%; for stage 2 it is about 60%; and for stage 3, it drops to about 30%. However, if treatment is delayed until stage 4, the 5-year survival rate is only 10%. The 5-year survival rate varies greatly among periods.

Therefore, if lung cancer can be detected early, it will be of great help in treating. Currently, there are several methods for testing lung cancer on the market:

1. Chest X-ray: It is more suitable for the detection of mid-to-late stage of lung cancer. Although it is not invasive and the examination is quite fast, it requires a large tumor to be observed through X-ray. Besides, due to the radiation, it is not easy to test at home.

2. Low-dose computed tomography (LDCT): It is more suitable for the detection of mid-to-late stage of lung cancer. Although it is not invasive and the examination is quite fast, due to the small amount of equipment in our country at present, an appointment is required. In addition, because the number of equipment is limited, it is also more expensive. Besides, it contains radiation like chest X-ray, and is not easy to test at home.

3. Next-generation sequencing (NGS): It is more suitable for precise treatment in the mid-to-late stages. Its sensitivity is much higher than other methods and can identify specific genes of lung cancer. However, because the equipment is more expensive and the operation is more complicated, it is not only time-consuming but also requires about 1-2 weeks to obtain results. In addition, it is also the most expensive of all methods.

4. Lung cancer tumor factor (Cyfra 21-1): It is more suitable for early tracking. Unfortunately, its sensitivity is not high, about 50% and takes several days to obtain the results. Besides, it is not suitable for home testing because of the complicated operation.

From the above description, it can be found that if we hope to have a higher 5-year survival rate for the treatment of lung cancer, early detection and early treatment are required. Unfortunately, the current early screening methods are not only insensitive, but also require a visit to a specific medical institution due to the complexity of the operation. It is not easy to perform testing at home. Besides, it takes several days to know the results.

Accordingly, providing a method of blood testing that is simple to operate, easy to detect at home, has higher sensitivity, and takes less time is a problem to be solved by those skilled in the art.

### SUMMARY OF THE INVENTION

An objective of the present invention is to provide a method that can test a target substance using a small amount of blood. After the blood is separated using a testing substrate, a spectrometer is used to detect the Raman spectrum of the blood amplified by the testing substrate. Then the concentration of the target substance can be detected. By comparing the spectrum with the pre-stored spectrum, a testing result can be generated.

To achieve the above objective, the present invention provides a method of testing blood, which comprises steps of: providing a testing substrate, the testing substrate comprising a substrate, a quantum well structure, a first-type doped semiconductor layer, a testing surface, and a plurality of nanometer metal particles located on the testing surface; disposing a blood sample on the testing substrate, the blood sample comprising a plurality of blood cells, the blood plasma, and a plurality of target substances; the testing substrate separating the blood sample to a first part and a second part, the first part comprising the plurality of blood cells, the blood plasma, and a portion of the plurality of target substrates, the second part comprising a portion of the plurality of blood cells and a portion of the plurality of target substances, with the number of the plurality of blood cells in the second part greater than the number of the plurality of blood cells in the first part; using a spectrometer to detect the Raman spectrum of the first part or the second part on the testing substrate for measuring the characteristics of the plurality of target substances and generating a target spectrum; and a control unit comparing the target spectrum and a preset spectrum for generating a testing result. By amplifying the Raman spectrum of the plurality of target substances in the blood sample by the testing substrate, the spectrometer can acquire the information of the plurality of target substances more clearly.

According to an embodiment of the present invention, in the step of using a spectrometer to detect the Raman spectrum of the first part or the second part on the testing substrate for measuring the characteristics of the plurality of target substances and generating a target spectrum, when the spectrometer detects the first part on the detection substrate, the plurality of target substances include β-carotene, DNA sequence, or tryptophan. When the spectrometer detects the second part on the detection substrate, the plurality of target substances include hemoglobin.

According to an embodiment of the present invention, in the step of the testing substrate separating the blood sample to a first part and a second part, the testing substrate separating the blood sample to a first part and a second part according to the different surface diffusion rates of the first part and the second part.

According to an embodiment of the present invention, in the step of using a spectrometer to detect the Raman spectrum of the first part on the testing substrate, the testing substrate amplifies the Raman spectrum of the plurality of target substances.

According to an embodiment of the present invention, in the step of disposing a blood sample on the testing substrate, the blood sample is stored in a refrigerator at −80 degrees Celsius, and the blood sample is 5 microliters of blood.

According to an embodiment of the present invention, in the step of providing a testing substrate, the testing surface is suitable for carrying a testing sample, so that the testing sample is disposed adjacent to the plurality of nanometer metal particles.

According to an embodiment of the present invention, in the step of providing a testing substrate, the material of the substrate comprises sapphire, silicon, or silicon carbide.

According to an embodiment of the present invention, in the step of providing a testing substrate, the material of the first-type doped semiconductor layer comprises gallium nitride or aluminum gallium nitride.

According to an embodiment of the present invention, in the step of providing a testing substrate, the quantum well structure comprises a plurality of first metal nitride layers and a plurality of second metal nitride layers.

According to an embodiment of the present invention, in the step of providing a testing substrate, in the quantum well structure, the number of the plurality of first metal nitride layers is between 1 and 15.

According to an embodiment of the present invention, in the step of providing a testing substrate, the testing substrate further comprises an undoped semiconductor layer disposed on the quantum well structure and the testing surface is located on the undoped semiconductor layer.

According to an embodiment of the present invention, in the step of providing a testing substrate, the material of the plurality of nanometer metal particles comprises metal, aluminum, silver, or copper.

### BRIEF DESCRIPTION OF DRAWINGS

FIG. 1 shows a flowchart according to an embodiment of the present invention;

FIG. 2 shows a system diagram of blood testing according to the present invention;

FIG. 3 shows a schematic diagram of the testing substrate according to the present invention;

FIG. 4 shows a schematic diagram of illuminating light to the testing substrate according to the present invention;

FIG. 5 shows a spectrum of the first part of blood sample according to the present invention;

FIG. 6 shows a spectrum of the second part of blood sample according to the present invention;

FIG. 7 shows the preset spectra according to the present invention;

FIG. 8 shows the preset spectrum according to the present invention;

FIG. 9 shows the preset spectra according to the present invention; and

FIG. 10 shows the preset spectra according to the present invention.

### DETAILED DESCRIPTION OF THE INVENTION

In order to make the structure and characteristics as well as the effectiveness of the present invention to be further understood and recognized, the detailed description of the present invention is provided as follows along with embodiments and accompanying figures.

In the specifications and subsequent claims, certain words are used for representing specific devices. A person having ordinary skill in the art should know that hardware manufacturers might use different nouns to call the same device. In the specifications and subsequent claims, the differences in names are not used for distinguishing devices. Instead, the differences in functions are the guidelines for distinguishing. In the whole specifications and subsequent claims, the word “comprising” is an open language and should be explained as “comprising but not limited to”. Besides, the word “couple” comprises any direct and indirect electrical connection. Thereby, if the description is that a first device is coupled to a second device, it means that the first device is connected electrically to the second device directly, or the first device is connected electrically to the second device via other device or connecting means indirectly.

The detection of lung cancer tumor factors according to the prior art not only takes several days, but also requires complex operations and can only be performed by professionals. Thereby, it is not conducive to early home testing and still has many shortcomings in early detection of cancer.

The present invention provides a blood testing method, which separates the blood sample by a testing substrate. The testing substrate amplifies the Raman spectrum of the target substance in the blood sample via the testing substrate, so that the spectrometer can better obtain the Raman spectrum of the target substance. By comparing to the preset spectrum, the testing result can be generated. The blood testing method according to the present invention is not only simple to operate, but also more convenient for home testing. The time required to generate the result is also much shorter than that of lung cancer tumor factors. Thereby, it can be more popularized and achieve early detection of diseases.

In the following description, various embodiments of the present invention are described using figures for describing the present invention in detail. Nonetheless, the concepts of the present invention can be embodied by various forms. Those embodiments are not used to limit the scope and range of the present invention.

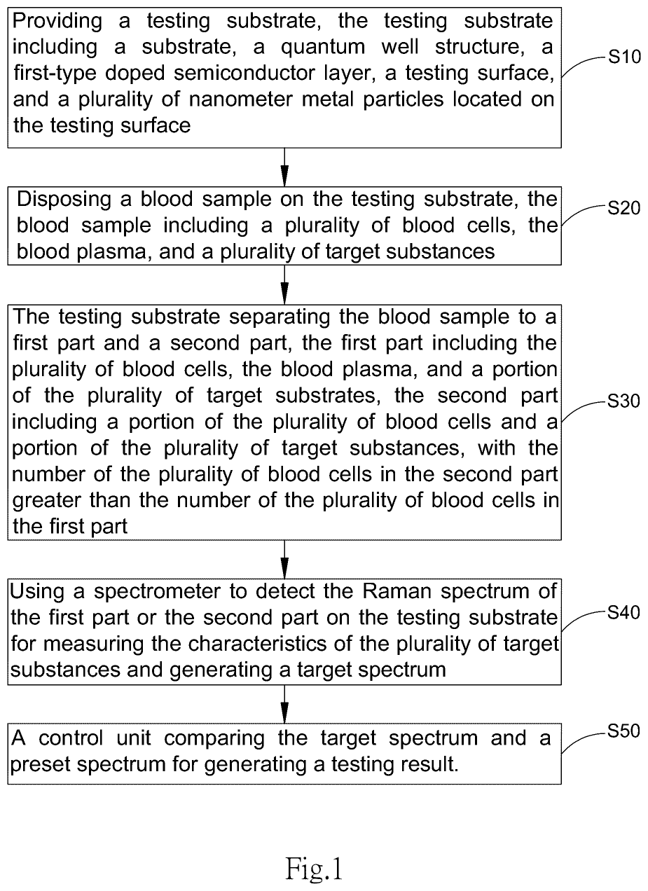

First, please refer to FIG. 1, which shows a flowchart according to an embodiment of the present invention. The embodiment of the present invention provides a blood testing method, which comprises the following steps of:

Step S10: Providing a testing substrate, the testing substrate comprising a substrate, a quantum well structure, a first-type doped semiconductor layer, a testing surface, and a plurality of nanometer metal particles located on the testing surface;

Step S20: Disposing a blood sample on the testing substrate, the blood sample comprising a plurality of blood cells, the blood plasma, and a plurality of target substances;

Step S30: The testing substrate separating the blood sample to a first part and a second part, the first part comprising the plurality of blood cells, the blood plasma, and a portion of the plurality of target substrates, the second part comprising a portion of the plurality of blood cells and a portion of the plurality of target substances, with the number of the plurality of blood cells in the second part greater than the number of the plurality of blood cells in the first part;

Step S40: Using a spectrometer to detect the Raman spectrum of the first part or the second part on the testing substrate for measuring the characteristics of the plurality of target substances and generating a target spectrum; and

Step S50: A control unit comparing the target spectrum and a preset spectrum for generating a testing result.

In the following, the above-mentioned steps will be described in detail.

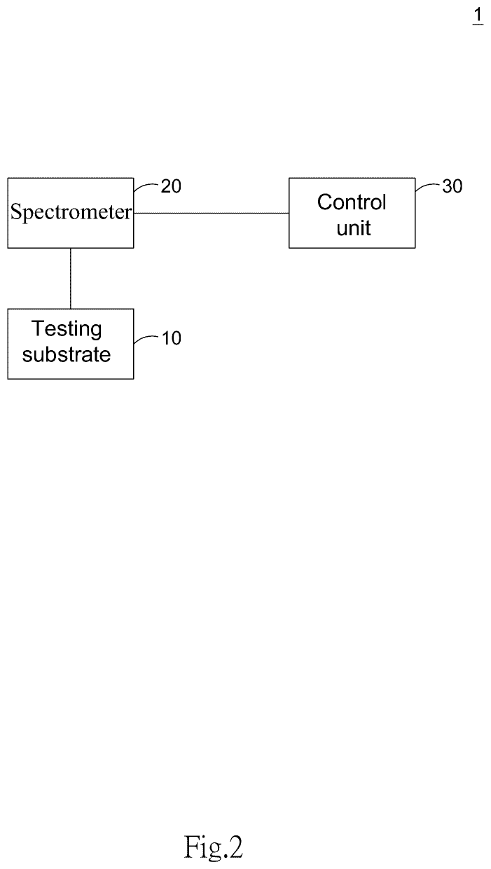

Please refer to FIG. 2, which shows a system diagram of blood testing according to the present invention. As shown in the figure, a blood testing system 1 comprises a testing substrate 10, a spectrometer 20, and a control unit 30. The spectrometer 20 is used for storing a preset spectrum, comparing it with the Raman spectrum, generating a testing result, and storing the testing result.

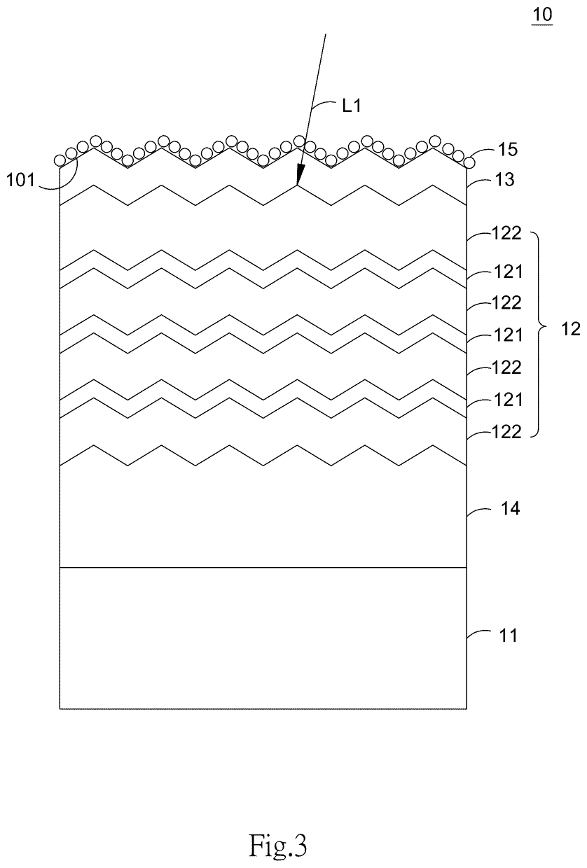

FIG. 3 shows a schematic diagram of the testing substrate according to the present invention. In the step S10, provide a testing substrate 10. The testing substrate 10 comprises a substrate 11, a quantum well structure 12, a first-type doped semiconductor layer 14. The first-type doped semiconductor layer 14 comprises a testing surface 101 and a plurality of nanometer metal particles 15. The plurality of nanometer metal particles 15 are located on the testing surface 101.

Please refer to FIG. 3. According to an embodiment, the testing substrate 10 comprises the substrate 11, the quantum well structure 12, and testing surface 101, and the plurality of nanometer metal particles 15. The quantum well structure 12 is disposed on the substrate 11. The plurality of nanometer metal particles 15 are disposed on the testing surface 101. The testing surface 101 according to the present embodiment is a rough surface. In other words, the testing surface 101 according to the present embodiment comprises multiple microstructures.

In the referenced FIG. 3 and the following figures, the plurality of nanometer metal particles 15 are represented by circular symbols of the same size, which are intended to illustrate the relative positions between these components and other components, and are not used to limit the size, shape, and position of the plurality of nanometer metal particles 15.

The quantum well structure 12 include a plurality of first metal nitride layers 121 and a plurality of second metal nitride layers 122. The plurality of first metal nitride layers 121 and the plurality of second metal nitride layers 122 are stacked alternately on the substrate 11.

The quantum well structure 12 according to the present embodiment is located between the testing surface 101 and the substrate 11. In addition, the location of the quantum well structure 12 is adjacent to the testing surface 101. When the testing surface 101 receives a first testing ray L1, the testing ray L1 will stimulate the quantum well structure 12 to make the quantum well structure 12 generate excess photons.

According to another embodiment, the material of the plurality of first metal nitride layer 121 of the quantum well structure 12 can include indium gallium nitride (InGaN), Aluminum gallium nitride (AlGaN), or gallium nitride (GaN).

According to another embodiment, the material of the plurality of first metal nitride layer 121 of the quantum well structure 12 can include gallium nitride (GaN), aluminum nitride (AlN), indium gallium nitride (InGaN), or Aluminum gallium nitride (AlGaN).

In the above embodiment, the quantum potential of the plurality of first metal nitride layers 121 is lower than the quantum potential of the plurality of second metal nitride layers 122. Thereby, the plurality of first metal nitride layers 121 can form multiple quantum well in the quantum well structure 12.

According to another embodiment, the material of the substrate 11 can include sapphire for providing a suitable surface for the growth these semiconductor layers. Nonetheless, the present invention is not limited to the embodiment. According to other embodiments, the material of the substrate 11 can include silicon or silicon carbide (SIC).

According to another embodiment, in the quantum well structure 12, the thickness of the plurality of first metal nitride layers 121 is a half of thickness of the plurality of second metal nitride layers 122.

According to another embodiment, in the quantum well structure 12, the number of the plurality of first metal nitride layers 121 is 3. Nonetheless, the present invention is not limited to the embodiment. The number of the plurality of first metal nitride layers 121 can be between 1 and 15. Consequently, the number of quantum wells in the quantum well structure 12 can be between 1 and 15.

Please refer to FIG. 3. According to another embodiment, the testing substrate 10 can further include the first-type doped semiconductor layer 140 disposed on the substrate 11. Besides, the first-type doped semiconductor layer 140 is located between the quantum well structure 12 and the substrate 11. The material of the first-type doped semiconductor layer 140 include GaN or AlGaN. In addition, the first-type doped semiconductor layer 140 is an n-type GaN layer. The first-type doped semiconductor layer 140 and the quantum well structure 12 can provide excellent heat dissipation capability between the testing substrate 10 and the testing surface 101 to avoid excessive overall temperature of the testing substrate 10. Moreover, the testing performance on the testing sample on the testing surface 101 will not be affected due to thermal diffusion of atoms or molecules.

In the step S20, dispose a blood sample 16 on the testing substrate 10, the blood sample 16 comprising a plurality of blood cells, the blood plasma, and a plurality of target substances

According to the present embodiment, the blood sample 16 only requires 5 μL of blood for testing, and the blood sample 16 is stored in a refrigerator at −80 degrees Celsius.

According to the present embodiment, the plurality of target substance are β-carotene. According to the research results of the National Cancer Institute, after smokers supplemented with high doses of β-carotene, the incidence of lung cancer was increased by 28%, and patients with lung cancer also have more β-carotene in their blood. Therefore, this embodiment provides relevant information about lung cancer by measuring β-carotene.

According to another embodiment, the target substance is a DNA sequence, which is used to detect various cancers by detecting the gene sequences of specific cancer cells.

According to another embodiment, the target substance is a hemoglobin, which is used to provide relevant information about bone hyperplasia tumors.

According to another embodiment, the target substance is a tryptophan, which is used to provide relevant information about bladder cancer.

In the step S30, the testing substrate 10 separates the blood sample 16 to a first part and a second part. The first part comprises a portion of the plurality of blood cells, the blood plasma, and the plurality of target substrates. The second part comprises a portion of the plurality of blood cells and a portion of the plurality of target substances. The number of the plurality of blood cells in the second part is greater than the number of the plurality of blood cells in the first part

The testing substrate 10 separates the blood sample 16 into the first part and the second part according to the different surface diffusion speed of the first part and the second part. Because the surface diffusion speed of the blood cells is slow, the blood cells in the second part are more than the blood cells in the first part. This process only takes 5 to 10 minutes.

In the step S40, use a spectrometer to detect the Raman spectrum of the first part or the second part on the testing substrate 10 for measuring the characteristics of the plurality of target substances and generating a target spectrum.

According to another embodiment, when the spectrometer 20 tests the first part on the testing substrate 10, the plurality of target substances include β-carotene, DNA sequence, or tryptophan. When the spectrometer 20 tests the second part on the testing substrate 10, the plurality of target substances include hemoglobin.

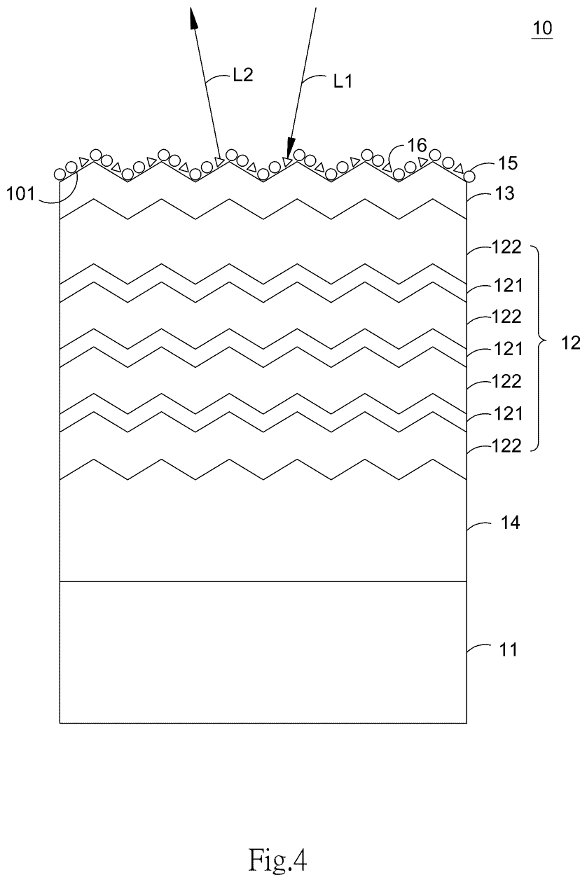

Please refer to FIG. 4, which shows a schematic diagram of illuminating light to the testing substrate according to the present invention. As shown in the figure, according to the present embodiment, because the testing surface 101 is a rough surface, it can carry more of the blood sample 16 and the plurality of nanometer metal particles 15. The blood sample 16 and the plurality of nanometer metal particles 15 can be placed on the testing surface 101 respectively, and creating more resonant intervals between each other. When the first testing ray L1 irradiates the testing surface 101, the blood sample 16 will receive the first testing ray L1 and generate a second testing ray L2. Then the first testing ray L1 can induce resonance in these resonance intervals. This phenomenon causes the blood sample 16 to generate stronger second testing light L2.

In the referenced FIG. 4 and the following figures, the plurality of nanometer metal particles 15 are represented by circular symbols of the same size, and the blood sample 16 is represented by a triangular symbol of the same size. The figures are intended to illustrate the relative positions of these elements, not to limit the size, shape, and position of the plurality of nanometer metal particles 15 and the blood sample 16.

According to the present embodiment, the second testing light L2 compris, for example, the Raman scattering light of the blood sample 16, and a qualitative measurement of the blood sample 16 can be made by analyzing the second testing light L2. On the other hand, since the testing substrate 10 comprises the quantum well structure 12, the first testing light L1 can stimulate the quantum well structure 12, causing the quantum well structure 12 to emit additional photons. The additional photons generated by the quantum well structure 12 can be transmitted to the blood sample 16 again, thereby increasing the intensity of the second testing light L2. On the other hand, by stimulating the quantum well structure 12 with the first testing light L1, the carrier concentration in the conduction band or other high-energy energy levels on the detection surface 101 of the testing substrate 10 will be increased as well. At the same time, the blood sample 16 on the testing surface 101 will also resonate with these carriers and then emit the Raman scattering light again. Therefore, the testing substrate 10 of this embodiment can greatly improve the testing efficiency of the blood sample 16 in all aspects through the quantum well structure 12, the plurality of nanometer metal particles 15, and the testing surface 101, so that the intensity of the second testing light L2 can be greatly increased.

According to another embodiment, the material of the plurality of nanometer metal particles 15 is gold, but the invention is not limited thereto. In other embodiments of the present invention, the material of the plurality of nanometer metal particles 15 may also include silver, aluminum, or copper. Therefore, the surface electrons of the plurality of nanometer metal particles 15 can resonate with the blood sample 16, and the plurality of nanometer metal particles 15 are suitable to generate localized surface plasma resonance (LSPR) with the blood sample 16. The plurality of nanometer metal particles 15 in this embodiment are formed by e-beam evaporator on the testing surface 101 to form a metal layer with a thickness of approximately 7 nanometers with the material of gold and annealing at 300 degrees Celsius for 140 seconds in a nitrogen ambient to form the plurality of nanometer metal particles 15. In this embodiment, the diameter of the plurality of nanometer metal particles 15 can be in the range of 10 nanometers to 100 nanometers.

Please refer to FIG. 4. According to the present embodiment, the testing substrate 10 further incudes an undoped semiconductor layer 13. The undoped semiconductor layer 13 is disposed on the quantum well structure 12; and the testing surface 101 is located on the undoped semiconductor layer 13. Furthermore, the undoped semiconductor layer 13 covers the quantum well structure 12; and the undoped semiconductor layer 13 has a thickness.

The thickness of the undoped semiconductor layer 13 is 6 nanometers. Therefore, the undoped semiconductor layer 13 can provide a protective function on the quantum well structure 12 and an appropriate spacing between the testing surface 101 and the quantum well structure 12 to increase the probability that the carriers on the testing surface 101 resonate with the blood sample 16, thereby increasing the intensity of the Raman scattering light in the second testing ray L2. On the other hand, the microstructures on the testing surface 101 of the testing substrate 10 can be formed by adjusting the air flow and temperature during the growth of the undoped semiconductor layer 13 to form a rough surface.

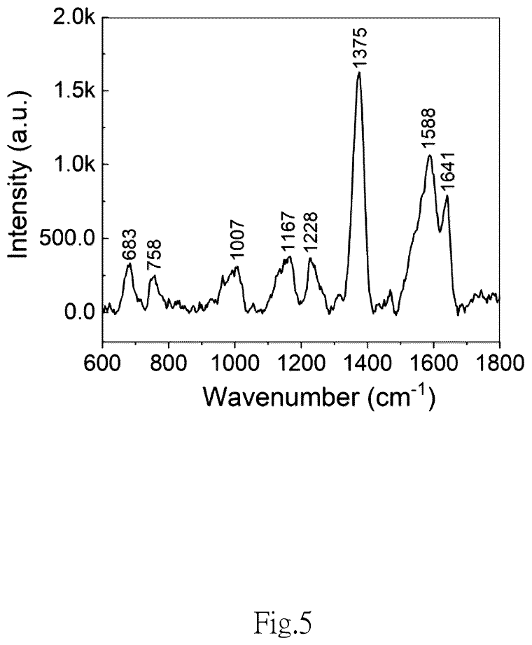

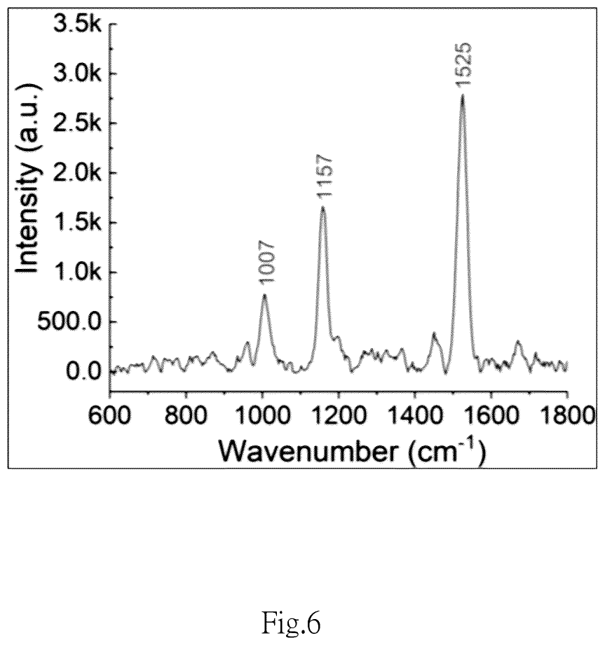

Please refer to FIG. 5 and FIG. 6. FIG. 5 shows a spectrum of the first part of blood sample according to the present invention; FIG. 6 shows a spectrum of the second part of blood sample according to the present invention. It can be seen that the spectrum of the second part of the blood sample 16 has three peaks, which represent the content of the β-carotene. The higher the peak value, the higher the β-carotene content contained in the blood sample 16.

In the step S50, a control unit 30 compares the target spectrum and a preset spectrum for generating a testing result.

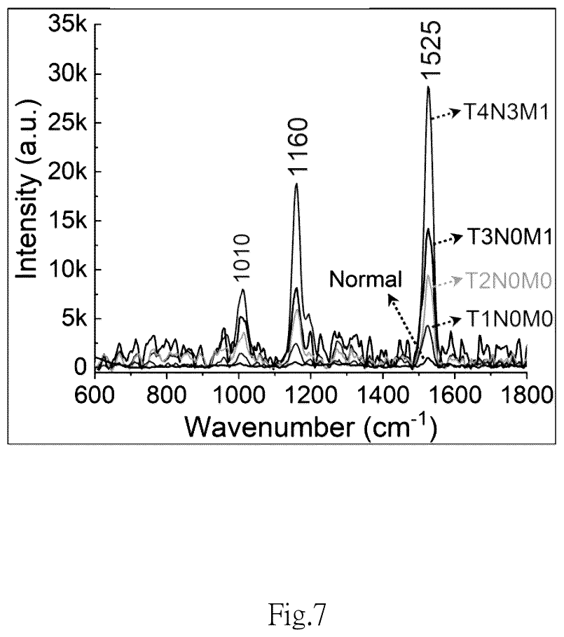

Please refer to FIG. 7, which shows the preset spectra according to the present invention. As shown in the figure, T1N0M0 represents the Raman spectrum of stage 1 lung cancer; T2N0M0 represents the Raman spectrum of stage 2 lung cancer; T3N0M1 represents the Raman spectrum of stage 3 lung cancer; and T4N3M1 represents the Raman spectrum of stage 4 lung cancer. It can be seen that a higher peak value of the target spectrum corresponds to a relatively late stage of lung cancer. In this embodiment, the control unit 30 compares with the preset spectrum and generates a testing result to determine whether the blood samples 16 have the possibility of lung cancer and the possible stage, thereby reminding the need for treatment or more in-depth examination.

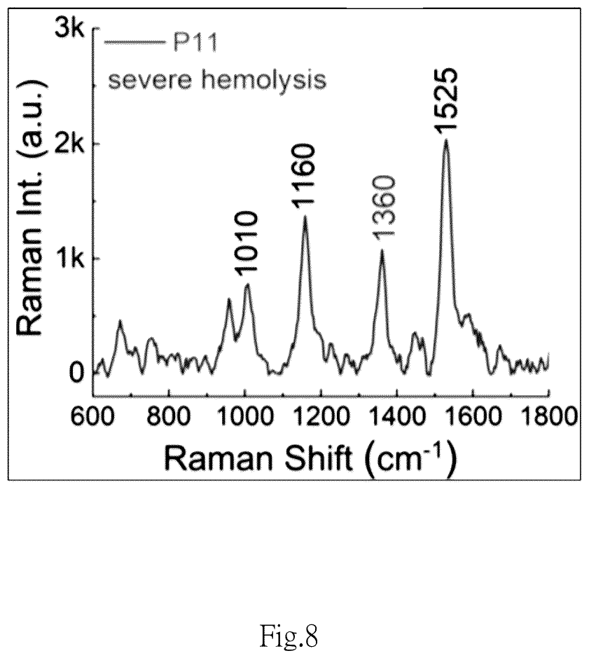

According to another embodiment, please refer to FIG. 8, which shows the preset spectrum according to the present invention. As shown in the figure, the peak of 1360 corresponds to the Raman spectrum of the hemoglobin. The higher the peak of the target spectrum, the higher the concentration of the hemoglobin that the blood sample 16 contains. In this embodiment, the control unit 30 compares with the preset spectrum and generates a testing result to determine whether the blood sample 16 has the possibility and possible stage of bone hyperplasia tumors, thereby reminding the need for treatment or more in-depth examination.

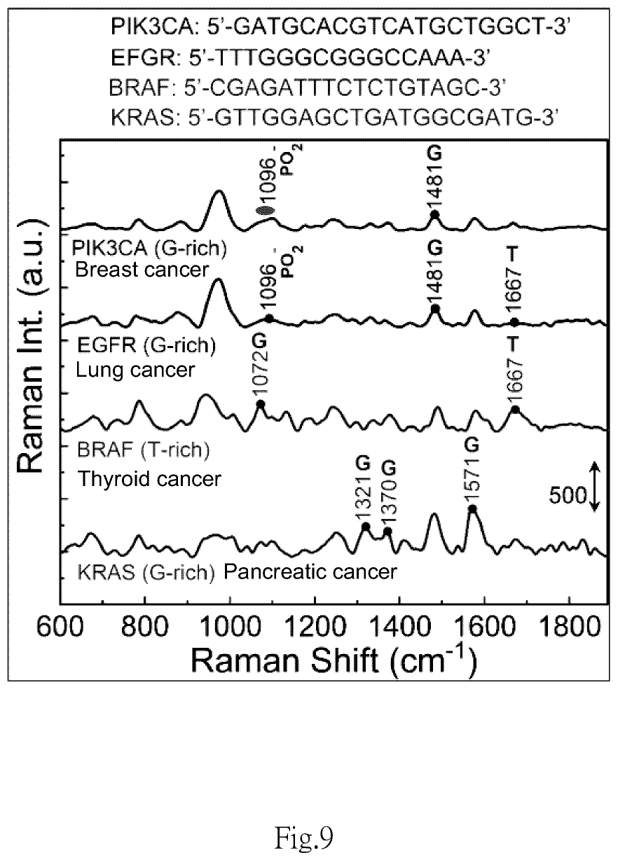

According to another embodiment, please refer to FIG. 9, which shows the preset spectra according to the present invention. As shown in the figure, the figure lists the gene sequences corresponding to the breast cancer, lung cancer, thyroid cancer, and pancreatic cancer, respectively. The breast cancer is 5′-GATGCACGTCATGCTGGCT-3′; the lung cancer is 5′-TTTGGGCGGGCCAAA-3′; the thyroid cancer is 5′-CGAGATTTCTCTGTAGC-3′; and the pancreatic cancer is 5′-GTTGGAGCTGATGGCGATG-3′. In this embodiment, after comparing with the preset spectrum, the control unit 30 generates a testing result to determine whether the blood samples 16 have breast cancer or lung cancer., thyroid cancer and pancreatic cancer, as well as the possible stages, to remind you of the need for treatment or more in-depth examinations. In this embodiment, the control unit 30 compares with the preset spectrum and generates a testing result to determine whether the blood sample 16 has the possibility and possible stage of the breast cancer, lung cancer, thyroid cancer, and pancreatic cancer, thereby reminding the need for treatment or more in-depth examination.

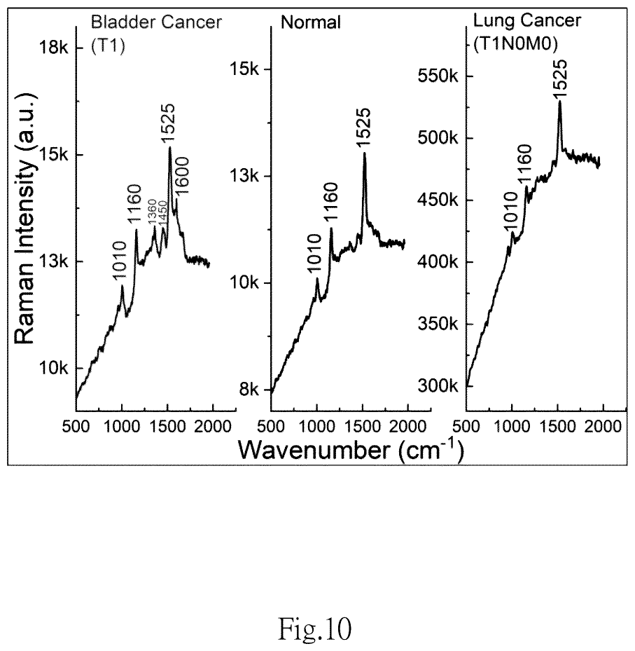

According to another embodiment, please refer to FIG. 10, which shows the preset spectra according to the present invention. As shown in the figure, the figure lists the preset spectra corresponding to the bladder cancer, a healthy subject, and the lung cancer, respectively. In this embodiment, it can be found that the preset spectra of the bladder cancer and the healthy subject have several peaks that are different. Among them, the peak value of 1600 corresponds to the Raman spectrum of tryptophan and can be used to determine whether the blood samples 16 have the possibility and possible stage of the bladder cancer through the preset spectrum, thereby reminding the need for treatment or more in-depth examination.

The present invention provides a blood testing method that uses a testing substrate to amplify the Raman spectrum of a specific band, so that a better spectrum presentation can be obtained through the spectrometer. In addition, the blood can be separated through the testing substrate, and at the same time enhance the efficacy of Raman spectrum. The blood testing method according to the present invention is much faster than the current testing method, which takes several days. Instead, the present invention takes about 30 minutes only to complete testing and provides the testing results. Moreover, it does not require a large amount of blood to perform the test, only 5 μL is required. Because of its simple operation, it is easier to test at home, providing early detection of various cancers, enabling early detection and early treatment, and improving the survival rate of cancer, which is undoubtedly a great benefit to human health.

Accordingly, the present invention conforms to the legal requirements owing to its novelty, nonobviousness, and utility. However, the foregoing description is only embodiments of the present invention, not used to limit the scope and range of the present invention. Those equivalent changes or modifications made according to the shape, structure, feature, or spirit described in the claims of the present invention are included in the appended claims of the present invention.

1. A method of testing blood, comprising steps of:
providing a testing substrate, comprising a substrate, a quantum well structure, a first-type doped semiconductor layer, a testing surface, and a plurality of nanometer metal particles located on said testing surface;
disposing a blood sample on said testing substrate, said blood sample comprising a plurality of blood cells, the blood plasma, and a plurality of target substances;
said testing substrate separating said blood sample to a first part and a second part, said first part comprising said plurality of blood cells, said blood plasma, and a portion of said plurality of target substrates, said second part comprising a portion of said plurality of blood cells and a portion of said plurality of target substances, with the number of said plurality of blood cells in said second part greater than the number of said plurality of blood cells in said first part;
using a spectrometer to detect the Raman spectrum of said first part or said second part on said testing substrate for measuring the characteristics of said plurality of target substances and generating a target spectrum; and
a control unit comparing said target spectrum and a preset spectrum for generating a testing result.

2. The blood testing method of claim 1, wherein said step of using a spectrometer to detect the Raman spectrum of said first part or said second part on said testing substrate for measuring the characteristics of said plurality of target substances and generating a target spectrum, when said spectrometer detects said first part on said testing substrate, said plurality of target substances include β-carotene, DNA sequence, or tryptophan, and when said spectrometer detects said second part on said detection substrate, said plurality of target substances include hemoglobin.

3. The blood testing method of claim 1, wherein said step of said testing substrate separating said blood sample to a first part and a second part, said testing substrate separating said blood sample to a first part and a second part according to the different surface diffusion rates of said first part and said second part.

4. The blood testing method of claim 1, wherein said step of using a spectrometer to detect the Raman spectrum of said first part on said testing substrate, said testing substrate amplifies the Raman spectrum of said plurality of target substances.

5. The blood testing method of claim 1, wherein said step of disposing a blood sample on said testing substrate, said blood sample is stored in a refrigerator at −80 degrees Celsius, and said blood sample is 5 microliters of blood.

6. The blood testing method of claim 1, wherein said step of providing a testing substrate, said testing surface is suitable for carrying a testing sample, so that said testing sample is disposed adjacent to said plurality of nanometer metal particles.

7. The blood testing method of claim 1, wherein said step of providing a testing substrate, the material of said substrate comprises sapphire, silicon, or silicon carbide.

8. The blood testing method of claim 7, wherein said step of providing a testing substrate, the material of said first-type doped semiconductor layer comprises gallium nitride or aluminum gallium nitride.

9. The blood testing method of claim 1, wherein said step of providing a testing substrate, said quantum well structure comprises a plurality of first metal nitride layers and a plurality of second metal nitride layers.

10. The blood testing method of claim 9, wherein said step of providing a testing substrate, in said quantum well structure, the number of said plurality of first metal nitride layers is between 1 and 15.

11. The blood testing method of claim 1, wherein said step of providing a testing substrate, said testing substrate further comprises an undoped semiconductor layer disposed on said quantum well structure and said testing surface is located on said undoped semiconductor layer.

12. The blood testing method of claim 1, wherein said step of providing a testing substrate, the material of said plurality of nanometer metal particles comprises metal, aluminum, silver, or copper.
